(19) United States
(12) Patent Application Publication
(10) Pub. No.: US 2024/0060137 A1
(43) Pub. Date: Feb. 22, 2024

(54) DETECTION SYSTEM AND DETECTION METHOD OF GENOMIC CARCINOGENESIS INFORMATION BASED ON CELL-FREE DNA

(71) Applicants: GENECAST BIOTECHNOLOGY CO., LTD., Wuxi (CN); GENECAST (BEIJING) BIOTECHNOLOGY CO., LTD., Beijing (CN)

(72) Inventors: Yulong LI, Beijing (CN); Yuanyuan HONG, Beijing (CN); Tiancheng HAN, Beijing (CN); Fang LV, Beijing (CN); Shunli YANG, Beijing (CN); Peiyao NIE, Beijing (CN); Qi ZHANG, Beijing (CN); Weizhi CHEN, Beijing (CN)

(21) Appl. No.: 18/052,067

(22) Filed: Nov. 2, 2022

Related U.S. Application Data
(63) Continuation of application No. PCT/CN22/98450, filed on Jun. 13, 2022.

(30) Foreign Application Priority Data
Jan. 7, 2022 (CN) 202210023902.1

(51) Int. Cl. C12Q 1/6886; G16B 20/10; G16B 20/30; G16B 30/00; G16H 50/30

(52) U.S. Cl. CPC C12Q 1/6886; G16B 20/10; G16B 20/30; G16B 30/00; G16H 50/30; C12Q 2600/154; C12Q 2600/156

(57) ABSTRACT

The present application provides a detection system and a detection method of genomic carcinogenesis information based on cell-free DNA, particularly plasma cell-free DNA. The system includes a library construction apparatus, a sequencing apparatus and an information analysis apparatus, the library construction apparatus is configured to convert 5-methylcytosine (5-mC) in the cell-free DNA in a to-be-detected sample into 5-formylcytosine (5-fC) and 5-carboxycytosine (5-caC) and convert non-methylated cytosine (C) into uracil (U) by using enzymes, and the information analysis apparatus is capable of analyzing methylation density of genome, fragment size distribution, fragment 5′ end motif and/or chromosome stability. With the adoption of the system and the method, early, sensitive and accurate detection and screening of various cancers can be synchronously implemented.

### CROSS-REFERENCE TO RELATED APPLICATIONS

The present application is a continuation of international application No. PCT/CN2022/098450, filed on Jun. 13, 2022, which claims priority to Chinese patent application No. 202210023902.1, filed Jan. 7, 2022, both of which are incorporated herein by reference in their entirety.

### TECHNICAL FIELD

The present invention relates to the field of genomic carcinogenesis information detection, and particularly relates to a detection system and a detection method of genomic carcinogenesis information based on cell-free DNA.

### BACKGROUND

Early screening and early diagnosis of cancers will provide possibility for timely treatment, and therefore the death rate of the cancers can be reduced. Traditional tumor diagnosis technologies focus on imaging examination such as gastroscopy and colonoscopy, the traditional tumor diagnosis technologies, as invasive detection means, may cause trauma to a patient, and the detection sensitivity is limited by the tumor development stage, only tumor lesions with the diameter larger than 1 cm can be found, and they are in the middle and later stages basically when being found. Pathological tissue biopsy is the gold standard of cancer diagnosis, but it is difficult to sample. Moreover, due to the heterogeneity of tumors, it is often difficult to realize complete sampling, which is not conductive to diagnostic classification, and easy to cause complications. A liquid biopsy technology, especially a technology for detecting biomarker signals of circulating tumor DNA (ctDNA) of tumor sources in cell-free DNA (cfDNA) in plasma, has been widely applied to tumor diagnosis, illness state tracking, relapse monitoring and the like as non-invasive tumor detection means in recent years. Compared with traditional imaging methods, the liquid biopsy technology has higher detection sensitivity on early tumors, can achieve simultaneous detection of multiple cancers, and has the potential of serving as a conventional cancer screening means for common population.

The ctDNA is derived from necrotic, apoptotic and circulating tumor cells as well as exosome secreted by the tumor cells, and carries genetic and epigenetic characteristics of the tumor cells. DNA methylation is an important apparent modification mode in eukaryotic cells, namely cytosine of a CpG island is converted into 5′-methylcytosine (5-mC) under the action of DNA methyltransferases (DNMTs). The change of the DNA methylation state is one of symbolic events in the tumor generation and development process, and it widely occurs in the genome at the early stage of the tumor. The CpG island in a human gene promoter region often has a high methylation phenomenon in cancer, which may silence the expression of certain cancer suppressor genes; and meanwhile, the cancer genome often presents a large-range demethylation state, so activation of a repeated sequence region or chromosome rearrangement may be caused.

A weak ctDNA signal will be sensitively detected by detecting the change of the plasma cfDNA methylation state. The human genome is greater than 3G, and for the consideration of sequencing cost, target region capture sequencing is the most common methylation detection means at present, but its performance is limited by screening of a cancer specific target region, and it is needed to perform high-depth whole-genome methylation sequencing analysis in the early stage on the cancer and a matched para-carcinoma tissue to select a differential methylation site. Therefore, the acquisition of various cancer high-quality tissue samples is a large bottleneck of the technical path, and the screening and verification processes of the differential methylation site are relatively tedious.

Except for the change of the methylation state, the fragmentation characteristics of the cfDNA of a cancer patient, including the proportion of fragments with different lengths in each region of the whole genome, fragment end sequences and the like, also show differences from healthy people, and in recent years, the fragmentation characteristics have been widely developed as another sensitive ctDNA epigenetic biomarker for detection of multiple cancers (“fragmentomics”). In addition, copy number variation (CNV) is a common genetic characteristic change in various cancers, and is also widely applied to detection of the ctDNA signals.

In a traditional methylation sequencing technology, non-methylated cytosine (C) is deaminized and converted into uracil (U) by utilizing bisulfite, and the high temperature and high pH environment of the reaction may cause serious degradation of DNA molecules, resulting in losing of original DNA fragment characteristics.

### SUMMARY

It is still needed to develop a system and a method which can analyze methylation, fragmentation characteristics, copy number variation and other characteristics at the same time for a single sequencing library constructed based on cell-free DNA, can detect genomic carcinogenesis information more accurately, sensitively, cheaply and easily; and the system and the method can be used for early, sensitive and accurate screening of various cancers at the same time.

The present invention is completed based on the following findings of the inventor: the inventor discovers for the first time that a sequencing library can be obtained by performing enzymatic treatment on plasma cfDNA (cell-free DNA) to convert 5-methylcytosine (5-mC) into 5-formylcytosine (5-fC) and 5-carboxycytosine (5-caC) and convert non-methylated cytosine (C) into uracil (U); and meanwhile, the sequencing library can be used for methylation and fragmentation of a whole genome (such as from two dimensions of fragment size index analysis and end motif analysis) and chromosome instability analysis (copy number variation), as well as early, sensitive and accurate screening of multiple cancers.

The present invention provides a library construction method and an analysis model which are low in cost and can simultaneously perform whole-genome methylation, fragmentation and copy number variation analysis on the plasma cfDNA to perform liquid biopsy screening of cancers. The method is suitable for low-initial-amount cfDNA, and target area capture is not needed, so that the technical process is simplified. Further, the detection sensitivity and accuracy of cancer screening can be further improved by optionally performing ensemble analysis on the cancer characteristics of all dimensions.

In one aspect, the present invention provides a detection system of genomic carcinogenesis information based on cell-free DNA (cfDNA), which includes:
a library construction apparatus, configured to convert 5-methylcytosine (5-mC) in the cell-free DNA (such as cell-free DNA in plasma) in a to-be-detected sample into 5-formylcytosine (5-fC) and 5-carboxycytosine (5-caC) and convert non-methylated cytosine (C) into uracil (U) by using enzymes to construct a library;
a sequencing apparatus, configured to sequence the constructed library; and
an information analysis apparatus, including one or more of the following modules:
a methylation analysis module, configured to analyze methylation information of the cell-free DNA,
a fragment size index analysis module, configured to analyze fragmentation information of the cell-free DNA,
an end motif analysis module, configured to analyze fragmentation information of the cell-free DNA, and
a chromosome instability analysis module, configured to analyze copy number variation information of chromosomes.

In some embodiments, the information analysis apparatus further includes an ensemble classification module, which is configured to perform ensemble on information obtained by the methylation analysis module, the fragment size index analysis module, the end motif analysis module and/or the chromosome instability analysis module.

In some embodiments, the methylation analysis module is an MD-KNN analysis module and is configured to divide human reference genome into bins (such as 1 Mb) in a non-overlapping sliding window method, calculate a proportion of methylation sites in all CpG sites of each bin, namely a methylation density (MD) value, and calculate a predicted value K of canceration possibility through a K-nearest neighbor (KNN) model.

In some specific embodiments, the fragment size index analysis module is an FSI-SVM analysis module and is configured to divide human reference genome into bins (such as 5 Mb) in a non-overlapping sliding window method, calculate a proportion of the number of short fragments (such as 101-167 bp) and the number of long fragments (such as 170-250 bp) in each bin to obtain a fragment size index (FSI) value of each sample, and calculate a predicted value F of canceration possibility through a support vector machine (SVM) model.

In some embodiments, the end motif analysis module is a Motif-SVM analysis module and is configured to calculate a proportion of 5 end 4-mer motif sequence of a fragment of the sample and calculate a predicted value S of canceration possibility through the SVM model.

In some embodiments, the chromosome instability analysis module is a CIN-PAscore analysis module and is configured to calculate a copy number of all semi-arm chromosomes of the sample, and calculate a plasma aneuploidy score (PAscore) by performing ensemble on z-scores of five semi-arm chromosomes with the maximum copy number variation of chromosomes corresponding to a healthy human baseline sample.

In some embodiments, the ensemble classification module is an SVM-ensemble classification module and is configured to perform ensemble on the predicted values K, F and S and the PAscore by using a linear SVM model to obtain a final predicted value Z of single canceration possibility.

In some specific embodiments, the library construction apparatus in the system includes:
a plasma cell-free DNA extraction module, configured to extract the cell-free DNA from a plasma sample;
an enzyme reaction module, configured to convert 5-methylcytosine (5-mC) in the cell-free DNA into 5-formylcytosine (5-fC) and 5-carboxycytosine (5-caC), and convert non-methylated cytosine (C) into uracil (U) by using enzymes; and
a PCR reaction module, configured to amplify the cell-free DNA subjected to enzyme reaction by using PCR.

In some specific embodiments, the used enzymes are TET2 enzyme and APOBEC enzyme.

In some specific embodiments, the sequencing apparatus is selected from Illumina Novaseq 6000, Illumina Nextseq500, MGIDNBSEQ-T7 or MGI SEQ-2000.

In some specific embodiments, the MD value in the MD-KNN analysis module is calculated through the following formula:

MD_{n,i}=Total_mC_{n,i}/Total_C_{n,i}
MD_{n,i} is the MD value of the i^{th} bin of sample n, Total_mCᵢ is the total number of all methylated C in the i^{th} bin, and Total_C_{n,i} is the total number of all C in the i^{th} bin.

In some specific embodiments, the FSI value in the FSI-SVM analysis module is calculated through the following formula:

FSI_{n,i}=Total_S_{n,i}/Total_L_{n,i}
FSI_{n,i} is the FSI value of the i^{th} bin of the sample n, Total_S_{n,i} is the number of short fragments in the i^{th} bin, and Total_L_{n,i} is the number of long fragments in the i^{th} bin.

In some specific embodiments, the proportion of motifs in the motif-SVM analysis module is calculated through the following formula:

$${Fraction}_{n , i} = {M}_{i} / {{\sum }_{i = 1}}^{256} {M}_{i}$$

Fraction_{n,i} is the proportion of the i^{th} 4-mer motif of the sample n, and Mᵢ is the number of the i^{th} 4-mer motifs.

In some specific embodiments, the PAscore in the CIN-PAscore analysis module is calculated through the following formula:

Z_{n,i}=(ARM_{n,i}−MEAN_baselineᵢ)/SD_baselineᵢ
Z_{n,i} is the z-score of semi-arm chromosome i of the sample n relative to the baseline sample, ARM_{n,i} is the reads number of the semi-arm chromosome i of the sample n, MEAN_baselineᵢ is the mean value of the reads number of the semi-arm chromosome i of the baseline sample, and SD_baselineᵢ is the standard deviation of the reads number of the semi-arm chromosome i of the baseline sample;
the z-scores of five semi-arm chromosomes with the maximum z-score absolute value of the to-be-detected sample n and the z-score of the semi-arm chromosome corresponding to the baseline sample are taken for subsequent analysis:

$$log {P}_{n} = {\sum }_{i = 1}^{5} [ - log ( dt ( {Z}_{n , i} , 3 ) ) ]$$

log Pₙ is a negative value of a logarithm sum of P values of the z-scores of the five semi-arm chromosomes of the sample n in t distribution with the degree of freedom being 3; and

PAscoreₙ=|log Pₙ−MEAN_baseline_{log P}|/SD_baseline_{log P}
PAscoreₙ is the PAscore of the sample n, MEAN_baseline_{log P} is the log P mean value of the baseline sample, and SD_baseline_{log P} is the standard deviation of the log P of the baseline sample.

In some specific embodiments, the information analysis apparatus includes a data preprocessing module which is configured to convert offline FASTQ data obtained by the sequencing apparatus into a Bam file which can be used by all modules and establish an index. For example, alignment, duplication elimination, sequencing and marking, screening and index establishing can be carried out.

In a second aspect, the present invention also provides a detection method of genomic carcinogenesis information based on cell-free DNA, which is performed by the system in the first aspect.

The detection method of genomic carcinogenesis information based on cell-free DNA includes:
library construction: converting 5-methylcytosine (5-mC) in the cell-free DNA (such as cell-free DNA in plasma) in a to-be-detected sample into 5-formylcytosine (5-fC) and 5-carboxycytosine (5-caC) and converting non-methylated cytosine (C) into uracil (U) by using enzymes to construct a library;
whole-genome sequencing: sequencing the constructed library; and
sequencing information analysis, including one or more of the following analysis steps:
methylation analysis: analyzing methylation information of the cell-free DNA,
fragment size index analysis: analyzing fragmentation information of the cell-free DNA,
end motif analysis: analyzing fragmentation information of the cell free DNA, and
chromosome instability analysis: analyzing copy number variation information of chromosomes.

In some specific embodiments, the sequencing information analysis further includes an ensemble classification step of performing ensemble on the information obtained through the methylation analysis, the fragment size index analysis, the end motif analysis and/or the chromosome instability analysis.

In some specific embodiments, the methylation analysis includes dividing human reference genome into bins (such as 1 Mb) in a non-overlapping sliding window method, calculating a proportion of methylation sites in all CpG sites of each bin, namely a methylation density (MD) value, and then calculating a predicted value K of canceration possibility through a KNN model, namely MD-KNN analysis for short.

In some specific embodiments, the fragment size index analysis includes dividing the human reference genome into bins (such as 5 Mb) in the non-overlapping sliding window method, calculating a proportion of the number of short fragments (such as 101-167 bp) and the number of long fragments (such as 170-250 bp) in each bin to obtain a fragment size index (FSI) value of each sample, and then calculating a predicted value F of the canceration possibility through an SVM model, namely FSI-SVM analysis.

In some specific embodiments, the end motif analysis includes calculating a proportion of a 5′ end 4-mer motif sequence of a fragment of the sample, and calculating a predicted value S of the canceration possibility through the SVM model, namely Motif-SVM analysis.

In some specific embodiments, the chromosome instability analysis includes calculating a copy number of all semi-arm chromosomes of the sample, and calculating PAscore by performing ensemble on z-scores of five semi-arm chromosomes with the maximum copy number variation of chromosomes corresponding to a healthy human baseline sample, namely CIN-PAscore analysis.

In some specific embodiments, the SVM-ensemble classification includes performing ensemble on the predicted values K, F and S and the PAscore by using a linear SVM model to obtain a final predicted value Z of single canceration possibility, namely SVM-ensemble classification.

In some specific embodiments, the library construction includes:
extracting the cell-free DNA (cfDNA) from a plasma sample;
enzyme reaction step, converting 5-methylcytosine (5-mC) in the cell-free DNA into 5-formylcytosine (5-fC) and 5-carboxycytosine (5-caC) and converting non-methylated cytosine (C) into uracil (U) by using enzymes; and
PCR amplification, amplifying the cell-free DNA subjected to enzyme reaction by utilizing PCR.

In some specific embodiments, the enzymes are TET2 enzyme and APOBEC enzyme.

In some specific embodiments, the sequencing is performed by using Illumina Novaseq 6000, Illumina Nextseq500, MGIDNBSEQ-T7 or MGI SEQ-2000.

In some specific embodiments, the MD value in the MD-KNN analysis module is calculated through the following formula:

MD_{n,i}=Total_mC_{n,i}/Total_C_{n,i}
MD_{n,i} is the MD value of the i^{th} bin of sample n, Total_mCᵢ is the total number of all methylated C in the i^{th} bin, and Total_C_{n,i} is the total number of all C in the i^{th} bin.

In some specific embodiments, the FSI value in the FSI-SVM analysis module is calculated through the following formula:

FSI_{n,i}=Total_S_{n,i}/Total_L_{n,i}
FSI_{n,i} is the FSI value of the i^{th} bin of the sample n, Total_S_{n,i} is the number of short fragments in the i^{th} bin, and Total_L_{n,i} is the number of long fragments in the i^{th} bin.

In some specific embodiments, the proportion of motifs in the motif-SVM analysis module is calculated through the following formula:

$${Fraction}_{n , i} = {M}_{i} / {{\sum }_{i = 1}}^{256} {M}_{i}$$

Fraction_{n,i} is the proportion of the i^{th} 4-mer motif of the sample n, and Mᵢ is the number of the i^{th} 4-mer motifs.

In some specific embodiments, the PAscore in the CIN-PAscore analysis module is calculated through the following formula:

Z_{n,i}=(ARM_{n,i}−MEAN_baselineᵢ)/SD_baselineᵢ
Z_{n,i} is the z-score of semi-arm chromosome i of the sample n relative to the baseline sample, ARM_{n,i} is the reads number of the semi-arm chromosome i of the sample n, MEAN_baselineᵢ is the mean value of the reads number of the semi-arm chromosome i of the baseline sample, and SD_baselineᵢ is the standard deviation of the reads number of the semi-arm chromosome i of the baseline sample;
the z-scores of five semi-arm chromosomes with the maximum z-score absolute value of the to-be-detected sample n and the z-score of the semi-arm chromosome corresponding to the baseline sample are taken for following analysis:

$$log {P}_{n} = {\sum }_{i = 1}^{5} [ - log ( dt ( {Z}_{n , i} , 3 ) ) ]$$

log Pₙ is a negative value of a logarithm sum of P values of the z-scores of the five semi-arm chromosomes of the sample n in t distribution with the degree of freedom being 3; and

PAscoreₙ=|log Pₙ−MEAN_baseline_{log P}|/SD_baseline_{log P}
PAscoreₙ is the PAscore of the sample n, MEAN_baseline_{log P} is the log P mean value of the baseline sample, and SD_baseline_{log P} is the standard deviation of the log P of the baseline sample.

In some specific embodiments, the information analysis further includes data preprocessing, including: converting offline FASTQ data obtained by a sequencing apparatus into a Bam file which can be used by all modules and establishing an index.

### BRIEF DESCRIPTION OF THE DRAWINGS

FIG. 1 shows a schematic flowchart of low-depth whole-genome sequencing and canceration information detection based on cfDNA according to the present invention.

FIG. 2A-2H show ROC curves of multiple cancer predication in an independent verification set performed by a KNN model (an MD-KNN analysis module) on whole-genome methylation density (MD) according to the present invention; wherein FIG. 2A shows a ROC curve of breast cancer predication, FIG. 2B shows a ROC curve of colorectal cancer predication, FIG. 2C shows a ROC curve of esophagus cancer predication, FIG. 2D shows a ROC curve of gastric cancer predication, FIG. 2E shows a ROC curve of liver cancer predication, FIG. 2F shows a ROC curve of lung cancer predication, FIG. 2G shows a ROC curve of pancreatic cancer predication, and FIG. 2H shows a ROC curve of entirety predication.

FIG. 3A-3H show ROC curves of multiple cancer predication in an independent verification set performed by an SVM model (an FSI-SVM analysis module) on whole-genome fragment size index (FSI) according to the present invention; wherein FIG. 3A shows a ROC curve of breast cancer predication, FIG. 3B shows a ROC curve of colorectal cancer predication, FIG. 3C shows a ROC curve of esophagus cancer predication, FIG. 3D shows a ROC curve of gastric cancer predication, FIG. 3E shows a ROC curve of liver cancer predication, FIG. 3F shows a ROC curve of lung cancer predication, FIG. 3G shows a ROC curve of pancreatic cancer predication, and FIG. 3H shows a ROC curve of entirety predication.

FIG. 4A-4H show ROC curves of multiple cancer predication in an independent verification set performed by an SVM model (a Motif-SVM analysis module) on fragment end characteristic motif proportion according to the present invention; wherein FIG. 4A shows a ROC curve of breast cancer predication, FIG. 4B shows a ROC curve of colorectal cancer predication, FIG. 4C shows a ROC curve of esophagus cancer predication, FIG. 4D shows a ROC curve of gastric cancer predication, FIG. 4E shows a ROC curve of liver cancer predication, FIG. 4F shows a ROC curve of lung cancer predication, FIG. 4G shows a ROC curve of pancreatic cancer predication, and FIG. 4H shows a ROC curve of entirety predication.

FIG. 5A-5H show ROC curves of multiple cancer predication in an independent verification set performed by PAscore measuring semi-arm chromosome instability (by a CIN-PAscore analysis module) according to the present invention; wherein FIG. 5A shows a ROC curve of breast cancer predication, FIG. 5B shows a ROC curve of colorectal cancer predication, FIG. 5C shows a ROC curve of esophagus cancer predication, FIG. 5D shows a ROC curve of gastric cancer predication, FIG. 5E shows a ROC curve of liver cancer predication, FIG. 5F shows a ROC curve of lung cancer predication, FIG. 5G shows a ROC curve of pancreatic cancer predication, and FIG. 5H shows a ROC curve of entirety predication.

FIG. 6A-6H show ROC curves of multiple cancer predication in an independent verification set performed by a final ensemble classification module according to the present invention; wherein FIG. 6A shows a ROC curve of breast cancer predication, FIG. 6B shows a ROC curve of colorectal cancer predication, FIG. 6C shows a ROC curve of esophagus cancer predication, FIG. 6D shows a ROC curve of gastric cancer predication, FIG. 6E shows a ROC curve of liver cancer predication, FIG. 6F shows a ROC curve of lung cancer predication, FIG. 6G shows a ROC curve of pancreatic cancer predication, and FIG. 6H shows a ROC curve of entirety predication.

### DETAILED DESCRIPTION

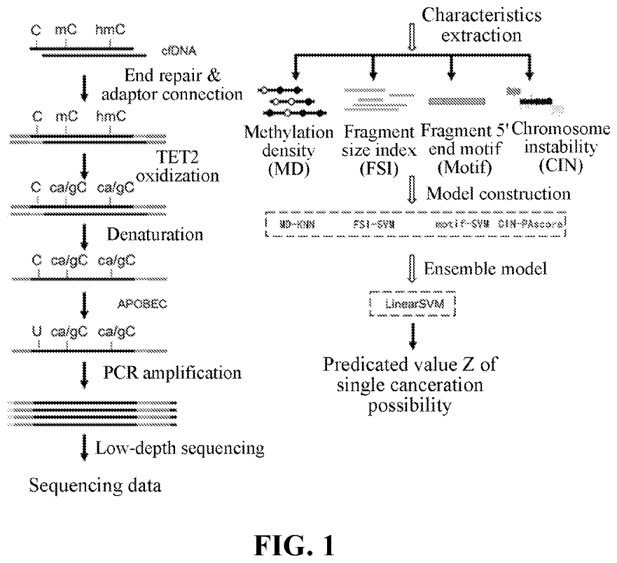

As shown in FIG. 1, the present invention includes low-depth fully-methylated whole-genome sequencing library construction and sequencing, where multi-dimensional characteristics extraction is performed on sequencing data, and a prediction model is constructed by machine learning.

### 1. Preparation of a cfDNA Fully-Methylated Whole-Genome Sequencing Library and Sequencing

### Principle:

In the present invention, TET2 enzyme and APOBEC enzyme are used for converting non-methylated cytosine (C) into uracil (U). Specifically, the TET2 enzyme is used for catalyzing 5-methylcytosine (5-mC) to be converted into 5-hydroxymethylcytosine (5-hmC), which is further oxidized into 5-formylcytosine (5-fC) and 5-carboxycytosine (5-caC), and thus 5-mC and 5-hmC are prevented from being acted in the subsequent APOBEC deamination reaction. Non-methylated cytosine (C) is deaminized and converted into uracil (U) by APOBEC enzyme, and uracil (U) is replaced by thymine (T) in the subsequent library amplification PCR reaction. Compared with a traditional bisulfite chemical reaction, reaction conditions of enzymatic conversion are mild, and the integrity of DNA molecules can be protected to the greatest degree, and therefore, enzymatic conversion can be used for analyzing cfDNA fragment characteristics and can also be used in library construction of low-initial-amount DNA.

### Solution:

1) cfDNA is extracted from 4 mL of serum of a healthy person or a cancer patient, and enzymatic conversion is performed on 5 ng to 30 ng of cfDNA by using TET2 and APOBEC to prepare a sequencing library.
2) Low-depth (about 20 G online data volume) 2×100PE sequencing is performed on the library.

### 2. Methylation Density (MD) Analysis

### Principle:

The methylation state in the tumor occurrence and development process may be abnormal in a large range in the genome. In the present invention, by comparing the similarity of methylation levels of a to-be-detected sample and a healthy person baseline in each region of the genome, whether the plasma methylation level is normal or not can be simply and sensitively determined, and then whether a ctDNA signal is contained or not can be speculated. In the analysis process, a machine learning algorithm can be used for modeling, and thus the detection sensitivity is further improved.

### Solution:

1) A human reference genome is divided into bins of 1 Mb in a sliding window method, and a proportion of methylation sites in all CpG sites of each bin is respectively calculated for each sample, namely methylation density (MD value).
2) A K-nearest neighbor (KNN) model is trained by utilizing the methylation density of a healthy person baseline and various cancer samples in a training set, and classification prediction of healthy persons or cancer patients is performed on to-be-tested samples in a test set by utilizing the KNN model.

### 3. Fragment Size Index (FSI) Analysis

### Principle:

The fragment size of cfDNA from tumor cells has greater heterogeneity than that of non-tumor cells. The FSI, namely a proportional map of a short fragment number and a long fragment number of cfDNA in each region of the whole genome, is highly consistent in healthy people, but will change in some regions of the cancer patients, which may reflect the abnormality of chromatin structures or other genome characteristics related to cancers. In the present invention, by comparing the cfDNA fragment size indexes of the to-be-detected sample and the healthy person baseline, whether ctDNA from the tumor exists or not can be simply and sensitively identified. Characteristics recognition can be carried out through the machine learning algorithm, and thus the detection sensitivity can be further improved.

### Solution:

1) The human reference genome is divided into bins of 5 Mb in a sliding window method, and a proportion of the number of short fragments and the number of long fragments in each bin is calculated for each sample to obtain the fragment size index of each sample.
2) A machine learning model is trained by utilizing the fragment size indexes of the healthy person baseline and various cancer samples in the training set, and an optimal model support vector machine (SVM) is selected to carry out classification prediction of healthy persons or cancer patients on the to-be-detected samples in the test set.

### 4. Fragment 5′ End Motif Analysis

### Principle:

4-mer motif sequence characteristics of a plasma cfDNA fragment end has preference, which may be related to sequence recognition characteristics of DNA endonucleases such as DNASE1L3. Abnormal expression may exist in related DNA endonucleases of the cancer patients, consequently, the cfDNA end sequence characteristics of the plasma of the cancer patients are changed, for example, the CCCA proportion is remarkably reduced in multiple cancers. In the present invention, 125 motif sequences with the highest proportion in 256 possible 4-mer motifs are selected, and the plasma end motif characteristics of the cancer patients are recognized through machine learning model training to determine the to-be-detected samples.

### Solution:

1) The proportion of 256 possible 4-mer motif sequences at the cfDNA fragment 5′ end of each sample is calculated. 125 motifs with the highest proportion in the healthy person baseline are selected.
2) The machine learning model is trained through the healthy person baseline and end motif frequency characteristics of various cancer samples in the training set, and an optimal model SVM is selected to carry out classification prediction of healthy persons or cancer patients on the to-be-detected samples in the test set.

### 5. Chromosome Instability (CIN) Analysis

### Principle:

Copy number variation is one of the most common genetic characteristic changes of cancer cells and is a common mechanism for cancer genome instability. The characteristics of most solid tumors include chromosome instability, which is represented as copy number change of the whole chromosome or part of chromosomes. In the present invention, the chromosome copy number of a semi-arm level is calculated and subjected to statistical analysis with the healthy person baseline, thus the chromosome variation of a tumor source can be directly identified, and a high-specificity liquid biopsy method is provided.

### Solution:

1) A reads number of each semi-arm chromosome is calculated.
2) Each semi-arm reads number of the to-be-detected sample is compared with the baseline sample, z-scores are calculated, five semi-arm chromosomes with the maximum z-score absolute value are selected, each z-score is converted into p-value and is subjected to ensemble to obtain a plasma aneuploidy score (PAscore) of the sample, thereby measuring the abnormality degree of the chromosome copy number of the sample.

### 6. Construction of an Ensemble Model Classifier (SVM-Ensemble Classification Module)

### Principle:

WMS data of each sample is analyzed in the above four dimensions, and whether the to-be-tested sample has a tumor signal can be comprehensively measured based on different biological mechanisms. An ensemble model is configured to perform ensemble on prediction results of the characteristics of each dimension to construct a classifier based on multi-component analysis, which can further improve the sensitivity and specificity of the model.

### Solution:

The machine learning model is trained by using the four-dimensional predicted values of the healthy human baseline and various cancer samples in the training set, an optimal model (linear SVM) is selected as the final ensemble classifier, and a final predicted value of single canceration possibility is calculated.

In addition to the foregoing advantages, compared with the related art, the present invention has many other advantages.

For example, in the present invention, abnormal methylation signals are recognized by detecting a plasma low-depth whole-genome methylation map; and compared with a common target zone capture sequencing method, utilizing cancer tissue or a public database to perform cancer difference methylation site screening and subsequent plasma cfDNA verification in advance is avoided, and therefore the methylation detection experiment and data analysis process is greatly simplified, and the detection cost is saved.

For example, in the present invention, methylation sequencing is carried out through an enzyme conversion method with mild reaction conditions, and compared with a bisulfite conversion method, the enzyme conversion method can reduce the damage to DNA molecules to the maximum degree. On one hand, this method is suitable for low-initial-amount cfDNA library construction, and the library can be successfully constructed only through cfDNA extracted from 10 mL of blood; and on the other hand, the original fragment characteristics of cfDNA molecules can be reserved through this method, and therefore ensemble analysis of methylation, fragment omics, CNV and other multi-dimensional characteristics can be carried out on the same cfDNA library, and thus the detection sensitivity and specificity are improved.

In another example, in the present invention, by directly comparing the similarity of genetic and epigenetic characteristics of the to-be-detected sample and the healthy person baseline in the whole-genome range, multiple cancers can be detected at the same time without screening different sites of various cancers.

### EXAMPLES

The solutions of the present invention are described below with reference to examples. Those skilled in the art may understand that the following examples are only used for describing the present invention and should not be construed as a limitation to the scope of the present invention. If the specific techniques or conditions are not indicated in the examples, the techniques or conditions described in the literature in the art or the product or instrument specification shall be followed. All reagents or instruments whose manufacturers are not given are commercially available.

### Clinical Cohort Sample Information:

Plasma of 497 healthy persons without cancer history and plasma of 795 cancer patients of multiple cancers at different cancer stages were selected retrospectively in this test and were randomly grouped into a training set and a verification set. The cancers of the patients included breast cancer, colorectal cancer, esophagus cancer, gastric cancer, liver cancer, lung cancer and pancreatic cancer. The training set included 352 healthy persons and 559 cancer patients (45 patients with breast cancer, 105 patients with colorectal cancer, 44 patients with esophagus cancer, 79 patients with gastric cancer, 79 patients with liver cancer, 110 patients with lung cancer, 83 patients with pancreatic cancer and 14 patients with other cancers), and 34.5% of the caners were at early stage (stage I or stage II). The verification set included 145 healthy persons and 236 cancer patients (21 patients with breast cancer, 45 patients with colorectal cancer, 18 patients with esophagus cancer, 35 patients with gastric cancer, 34 patients with liver cancer, 47 patients with lung cancer and 36 patients with pancreatic cancer), and 31.8% of the cancers were at early stage (stage I or stage II).

### I. Experiment Processes

### 1. Extraction of Plasma cfDNA

1.1 10 mL of whole blood of each subject was stored in a KANGWAY EDTA blood collection tube, and centrifugation was performed at 1600 g under 4° C. for 10 min to layer plasma and blood cells. The upper-layer plasma was transferred to a new centrifuge tube, then centrifugation was performed again at 12000 rpm under 4° C. for 15 min, and supernatant was collected to remove cell debris. About 4 mL of the plasma was obtained and frozen at −80° C. for later use.
1.2 After a plasma sample was melted, 15 μL of Proteinase K (20 mg/mL, thermoscientific cat #EO0492) and 50 μL of SDS (20%) were added into each 1 mL of the sample. In a case that the plasma amount was less than 4 mL, PBS was used for supplementing.
1.3 The sample was overturned and uniformly mixed, and incubated at 60° C. for 20 min, and then subjected to ice bath for 5 min.
1.4 cfDNA was extracted by a MagMAX Cell-Free DNA Isolation kit (thermoscientific cat #A29319).
1.5 The extraction concentration and quality of the cfDNA were detected by a Bioanalyzer 2100 (Agilent Technologies).

### 2. cfDNA Library Construction

A methylation library construction kit NEBNext Enzymatic Methyl-seq Kit (NEB, cat #E7120) was utilized, 5-30 ng of cfDNA was an initial amount, 5-methylcytosine (5-mC) was converted into 5-formylcytosine (5-fC) and 5-carboxycytosine (5-caC) by TET2 enzyme, non-methylated cytosine (C) was deaminized into uracil (U) by APOBEC enzyme, and then amplification library construction was performed.

The specific library construction process was as follows:

### 2.1 Preparation of Internal Reference

50 μL of CpG fully-methylated pUC19 DNA and 50 μL of CpG fully-non-methylated Lamdba DNA were uniformly mixed and then added into a 100 μL of breaking tube, and was broken by an M220 breaker (Covaris). During library construction, 0.001 ng of pUC19 DNA and 0.02 ng of lambda DNA were added into to-be-detected cfDNA.

### 2.2 Preparation of cfDNA Sample

An initial amount of the cfDNA sample was 5-30 ng, and breaking was not needed.

### 2.3 End Repair

2.3.1 The following reaction systems were mixed on ice;

| Reagent | Volume | |
|---|---|---|
| cfDNA Sample (5-30 ng) | 50 | μL |
| NEBNext Ultra II End Prep Reaction Buffer | 7 | μL |
| NEBNext Ultra II End Prep Enzyme Mix | 3 | μL |
| Total volume | 60 | μL |

2.3.2 The reaction systems were placed on a PCR instrument and subjected to end repair reaction according to the following table.

| | | |
|---|---|---|
| Step | Temperature | Time |

| | | | |
|---|---|---|---|
| End repair and add A tail | 20° | C. | 30 min |
| | 65° | C. | 30 min |
| Termination | 4° | C. | ∞ |

### 2.4 Adaptor Connection

2.4.1 The following components were added into the above 60 μL reaction system on ice.

| Reagent | Volume | |
|---|---|---|
| NEBNext EM-seq Adaptor | 2.5 | μL |
| NEBNext Ultra II Ligation Master Mix | 30 | μL |
| NEBNext Ligation Enhancer | 1 | μL |
| Total volume | 93.5 | L |

2.4.2 Incubation was performed at 20° C. for 15 min.

### 2.5 Purification After Connection

2.5.1 After the previous reaction was finished, the sample was taken out, 110 μL of NEBNext Sample Purification Beads was added and immediately uniformly mixed by blowing and beating through a pipettor.
2.5.2 Incubation was performed at room temperature for 5 min.
2.5.3 A centrifuge tube was placed on a magnetic frame for 5 min until the liquid was clarified, and then the supernatant was removed.
2.5.4 200 μL of 80% ethanol prepared freshly was added and incubated for 30 s and then removed. The step of cleaning with 200 μL of 80% ethanol was repeated once.
2.5.5 Residual ethanol at the bottom of the centrifuge tube was completely absorbed by a 10 μL pipettor and dried at room temperature for 3-5 min until the ethanol completely volatilized.
2.5.6 The centrifuge tube was taken down from the magnetic frame, and 29 μL of Elution Buffer (NEB) was added and oscillated to be uniformly mixed. Incubation was performed at room temperature for 1 min.
2.5.7 Centrifugation was performed temporarily, the centrifuge tube was placed on the magnetic frame for 3 min until the liquid was clarified, and 28 μL of liquid was transferred into a new PCR tube.

### 2.6 Oxidation Reaction of 5-Methylcytosine and 5-Hydroxymethylcytosine

The NEBNext Enzymatic Methyl-seq Kit (NEB, cat #E7120) was used in the following reaction operations.
2.6.1 TET2 Reaction Buffer Supplement dry powder was added into 400 μL of TET2 Reaction Buffer and fully mixed.
2.6.2 The following components were added into 28 μL of DNA with the adaptor connected on ice.

| Reagent | Volume | |
|---|---|---|
| TET2 Reaction Buffer (prepared in 2.6.1) | 10 | μL |
| DTT | 1 | μL |
| Oxidation Supplement | 1 | μL |
| Oxidation Enhancer | 1 | μL |
| TET2 | 4 | μL |
| Total volume | 17 | μL |

2.6.3 500 mM of Fe(II) solution was diluted according to a ratio of 1:1250. The prepared Fe(II) was added into the previous uniformly mixed product.

| Reagent | Volume | |
|---|---|---|
| DNA Sample | 45 | μL |
| Diluted Fe(II) | 5 | μL |
| Total volume | 50 | μL |

The materials were fully mixed and incubated at 37° C. for 1 h.
2.6.4 After the reaction was finished, the product was transferred to ice, and 1 μL of Stop Reagent was added.

| Reagent | Volume |
|---|---|
| Stop Reagent | 1 μL |
| Total volume | 51 μL |

The materials were fully mixed.
2.6.5 Incubation was performed at 37° C. for 30 min.

| Step | Temperature | Time |
|---|---|---|
| Terminate oxidization reaction | 37° C. | 30 min |

### 2.7 Purification After Oxidization

2.7.1 After previous reaction was finished, the sample was taken out, 90 μL of NEBNext Sample Purification Beads was added and immediately uniformly mixed by blowing and beating through the pipettor.
2.7.2 Incubation was performed at room temperature for 5 min.
2.7.3 The centrifuge tube was placed on the magnetic frame for 5 min until the liquid was clarified, and then the supernatant was removed.
2.7.4 200 μL of 80% ethanol prepared freshly was added and incubated for 30 s and then removed. The step of cleaning with 200 μL of 80% ethanol was repeated once.
2.7.5 Residual ethanol at the bottom of the centrifuge tube was completely absorbed by a 10 μL pipettor and dried at room temperature for 3-5 min until the ethanol completely volatilized.
2.7.6 The centrifuge tube was taken down from the magnetic frame, 17 μL of Elution Buffer was added and oscillated to be uniformly mixed. Incubation was performed at room temperature for 1 min.
2.7.7 Centrifugation was performed temporarily, the centrifuge tube was placed on the magnetic frame for 3 min until the liquid was clarified, and 16 μL of liquid was transferred into a new PCR tube.

### 2.8 DNA Denaturation

2.8.1 Fresh 0.1 N NaOH was prepared.
2.8.2 The PCR instrument was preheated to 50° C. in advance.
2.8.3 4 μL of 0.1 N NaOH was added into the 16 μL of purified product obtained in the previous step and fully mixed.
2.8.4 Incubation was performed at 50° C. for 10 min.
2.8.5 The product was immediately put on ice after the reaction was finished.

### 2.9 Cytosine Deamination

2.9.1 The following components were added into 20 μL of denatured DNA obtained in the previous step on ice.

| Reagent | Volume | |
|---|---|---|
| Nuclease-free water | 68 | μL |
| APOBEC Reaction Buffer | 10 | μL |
| BSA | 1 | μL |
| APOBEC | 1 | μL |
| Total volume | 80 | μL |

The materials were fully mixed.
2.9.2 Incubation was performed on the PCR instrument at 37° C. for 3 h, and the reaction was terminated at 4° C.

### 2.10 Purification After Deamination

2.10.1 After previous reaction was finished, the sample was taken out, 100 μL of NEBNext Sample Purification Beads was added and immediately uniformly mixed by blowing and beating through the pipettor.
2.10.2 Incubation was performed at room temperature for 5 min.
2.10.3 The centrifuge tube was placed on the magnetic frame for 5 min until the liquid was clarified, and then the supernatant was removed.
2.10.4 200 μL of 80% ethanol prepared freshly was added and incubated for 30 s and then removed. The step of cleaning with 200 μL of 80% ethanol was repeated once.
2.10.5 The residual ethanol at the bottom of the centrifuge tube was completely absorbed by the 10 μL pipettor and dried at room temperature for 3-5 min until the ethanol completely volatilized.
2.10.6 The centrifuge tube was taken down from the magnetic frame, 21 μL of Elution Buffer was added and oscillated to be uniformly mixed. Incubation was performed at room temperature for 1 min.
2.10.7 Centrifugation was performed temporarily, the centrifuge tube was placed on the magnetic frame for 3 min until the liquid was clarified, and 20 μL of liquid was transferred into a new PCR tube.

### 2.11 Library PCR Amplification

2.11.1 The following components were added into 20 μL of deaminated DNA obtained in the previous step on ice.

| Reagent | Volume | |
|---|---|---|
| EM-seq Index Prime | 5 | μL |
| NEBNext Q5U Master Mix | 25 | μL |
| Total volume | 30 | μL |

2.11.2 The fully-mixed materials were subjected to the following PCR reaction on the PCR instrument.

| | | | |
|---|---|---|---|
| Step | Temperature | Time | Cycle number |

| | | | | | |
|---|---|---|---|---|---|
| Pre-denaturation | 98° | C. | 30 | sec | 1 |
| Denaturation | 98° | C. | 10 | sec | 4-8 |
| Annealing | 62° | C. | 30 | sec | |
| Extension | 65° | C. | 60 | sec | |
| Re-extension | 65° | C. | 5 | min | 1 |

| | | | | |
|---|---|---|---|---|
| Storage | 4° | C. | ∞ | 1 |

### 2.12 Purification After PCR

2.12.1 After the previous reaction was finished, the sample was taken out, 45 μL of NEBNext Sample Purification Beads was added and immediately uniformly mixed by blowing and beating through the pipettor.
2.12.2 Incubation was performed at room temperature for 5 min.
2.12.3 The centrifuge tube was placed on the magnetic frame for 5 min until the liquid was clarified, and then the supernatant was removed.
2.12.4 200 μL of 80% ethanol prepared freshly was added and incubated for 30 s and then removed. The step of cleaning with 200 μL of 80% ethanol was repeated once.
2.12.5 The residual ethanol at the bottom of the centrifuge tube was completely absorbed by the 10 μL pipettor and dried at room temperature for 3-5 min until the ethanol completely volatilized.
2.12.6 The centrifuge tube was taken down from the magnetic frame, 21 μL of Elution Buffer was added and oscillated to be uniformly mixed. Incubation was performed at room temperature for 1 min.
2.12.7 Centrifugation was performed temporarily, the centrifuge tube was placed on the magnetic frame for 3 min until the liquid was clarified, and 20 μL of liquid was transferred into a new PCR tube.

### 2.13 Library Quantification

The constructed library was quantified by a Qubit high-sensitivity reagent (thermoscientific cat #Q32854), and subsequent online sequencing was performed when the library yield was greater than 400 ng.

### 3. Library Sequencing

10% PhiX DNA (Illumina cat #FC-110-3001) was added into 100 ng of the library and mixed to obtain an online sample, and PE100 sequencing was performed on a Novaseq 6000 (Illumina) platform.

### II. Bioinformatic Analysis Process

### 1. Process Offline FASTQ Data into a Bam File which can be Used by All Modules

### 1.1 Removal of Adaptor

Trimmomatic-0.36 was called to align each pair of FASTQ files as paired reads to an hgl9 human reference genome sequence, and an initial bam file was generated by using M parameter and an ID of a specified Reads Group, the other parameter options were not used.

### 1.2 Alignment

Bismark-v0.19.0 was called to align each pair of FASTQ files subjected to adaptor removal as paired reads to the hgl9 human reference genome sequence and a Lambda DNA reference genome sequence to generate an initial Bam file.

### 1.3 Deduplication

A deduplicate module of the Bismark-v0.19.0 was called to perform deduplication processing on the initial Bam file, so as to generate a deduplicated Bam file.

### 1.4 Sorting and Marking

A sort module of SAMtools-1.3 was called to sort the deduplicated Bam file, so as to generate a sorted Bam file. Then an AddOrReplaceReadGroups module of Picard-2.1.0 was called to mark and group the sorted Bam file.

### 1.5 Screening

A clipOverlap module of BamUtil-1.0.14 was called to screen the marked and grouped Bam file, so as to remove overlapped paired reads and generate the Bam file. SAMtools-1.3 view was called to filter the alignment quality of the overlapping-removed Bam file, and a final Bam file was generated by adopting “-q 20” as a parameter.

### 1.6 Index Establishment

An index module of the SAMtools-1.3 was called to establish an index for the finally generated Bam file, so as to generate a bai file paired with the final Bam file.

### 2. Methylation Density (MD) Analysis (MD-KNN Analysis Module)

2.1 The human reference genome was divided into bins of 1 Mb in a non-overlapping sliding window mode; 1846 bins were remained after bins with poor alignment rate were removed; a proportion of methylation sites in all CpG sites of the 1846 bins was respectively calculated for each sample, and this value corresponded to the MD value of each sample; and the specific formula was as follows:

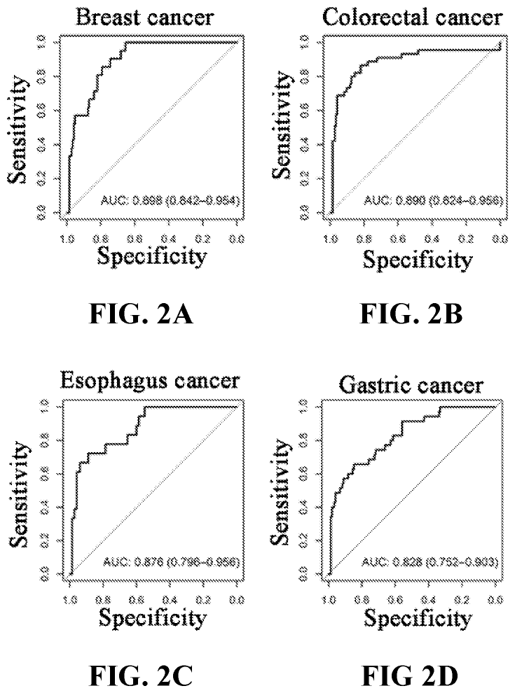

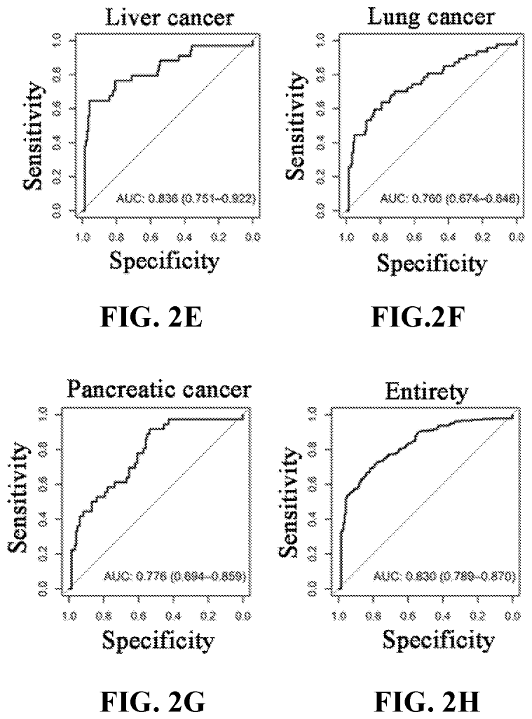

MD_{n,i}=Total_mC_{n,i}/Total_C_{n,i}
MD_{n,i} is the MD value of the i^{th} bin of the sample n, Total_mCᵢ is the total number of all methylated C in the i^{th} bin, and Total_C_{n,i} is the total number of all C in the i^{th} bin.
2.2 1846 MD values of each sample obtained in the step 2.1 were subjected to standardized processing to calculate z-scores, a Euclidean distance between the samples was calculated through a philentropy packet of R language, and 1/distance was selected for the weight of the samples. A parameter K was simulated and adjusted by 50 rounds, 80% of training set samples was used in each round; AUC was calculated according to a prediction result of 20% of samples of out-of-bag (OOB) of each round in the 50 rounds when K was at different values, and the K value with the highest AUC of the OOB sample was selected.
2.3 Classification prediction of healthy persons or cancer patients was performed on each to-be-detected sample in a test set by using a trained K-nearest neighbor (KNN) model to obtain a predicted value K. As shown in FIG. 2A-2H, the ROC curve area (AUC) of an MD-KNN classifier for detecting single cancer in the test set reached 0.789-0.870, the AUC performance for detecting all seven cancers reached 0.830, and thus good cancer detection performance was shown.

### 3. Fragment Size Index (FSI) Analysis (FSI-SVM Analysis Module)

3.1 The human reference genome was divided into bins of 5 Mb in a non-overlapping sliding window mode; 502 bins were remained after blacklist bins with poor alignment rate were removed; a proportion of the number of short fragments (101-167 bp) and the number of long fragments (170-250 bp) in the 502 bins was respectively calculated, and an LOESS algorithm was used for GC correction to obtain the FSI of each sample. The specific calculation formula was as follows:

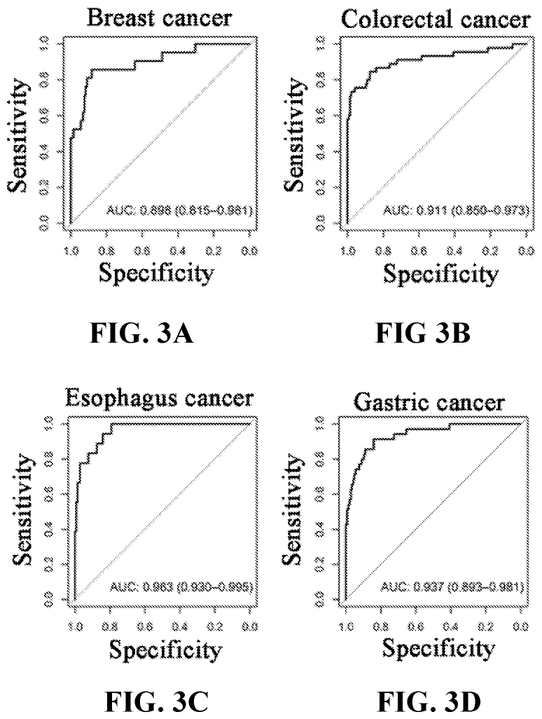

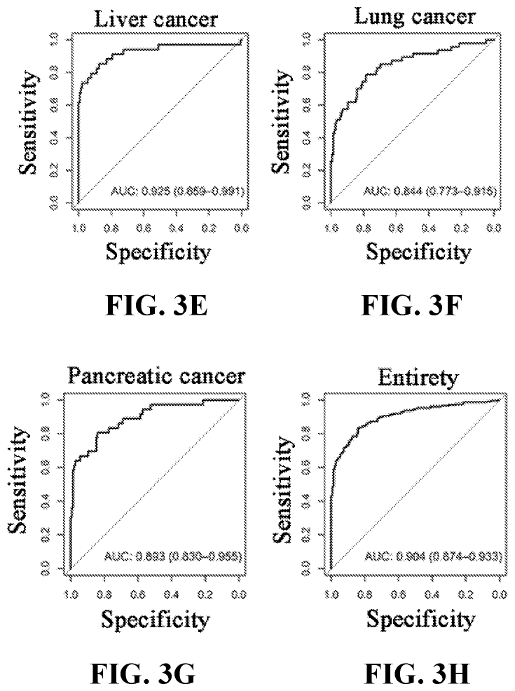

FSI_{n,i}=Total_S_{n,i}/Total_L_{n,i}
FSI_{n,i} is the FSI value of the i^{th} bin of the sample n, Total_S_{n,i} is the number of short fragments in the i^{th} bin, and Total_L_{n,i} is the number of long fragments in the i^{th} bin.
3.2 A support vector machine (SVM) model was trained by a sklearn packet of python for the 502 FSI values of each sample, hyper-parameters were selected by using a grid search mode, and 10-time cross validation was carried out to obtain the hyper-parameters.
3.3 Classification prediction of healthy persons or cancer patients was performed on each to-be-detected sample in the test set to obtain a predicted value F. As shown in FIG. 3A-3H, the ROC curve area (AUC) of an FSI-SVM classifier for detecting single cancer in the test set reached 0.874-0.933, the AUC performance for detecting all seven cancers reached 0.904, and thus good cancer detection performance was shown.

### 4. Fragment End Motif Analysis (Motif-SVM Analysis Module)

4.1 A proportion of 256 (namely possible permutation and combination of four basic groups, fourth power of 4) possible 4-mer motif sequences at the 5′ end of a fragment of each sample was calculated. 125 motifs with the proportion exceeding 0.0004 and having the highest proportion in the healthy person baseline were selected, as shown in the following Table 1.

TABLE 1

| | | | | |
|---|---|---|---|---|
| CCCA | CCTA | TGGA | TGCC | CTGT |
| CCTG | GGCT | TGAT | GGAT | CTAA |
| CCAG | CAGG | ACAA | ACTG | TCCC |
| CCCT | TATT | CAAT | GACA | TGGC |
| TAAA | GCAG | GCAA | GAAT | CAGT |
| CAAA | CACA | ACCA | TAGA | TCTA |
| CCAA | CATT | ACTT | GCAT | CTTG |
| CCTT | CAAG | TCTG | TCCA | TGAC |
| AAAA | CAGA | GCCC | TTTT | TGGT |
| GGAG | CTTT | ACCT | TACT | GGTC |
| CCAT | TGGG | GGCC | TCAG | TCAC |
| CCTC | CATG | ACAG | GCTC | AAGA |
| GCCT | TAAT | TCTC | CATC | CAAC |
| TGAA | TATA | CTGA | GGGG | ACCC |
| GCCA | TGCA | TATG | TCAT | CTTA |
| TGAG | TACA | CATA | CACC | TATC |
| CCAC | TGTA | ACAT | GAGA | ACAC |
| CCCC | TCTT | TAAG | GCTA | CTTC |
| GGTG | GGGA | AGAG | GGTA | GGGC |
| GCTG | GCTT | TCCT | GCAC | AGCA |
| GAAA | AAAT | CTGG | GAGG | AGGA |
| TGTT | AGAA | CAGC | AACA | GATG |
| GGAA | GAAG | CACT | TCAA | GATT |
| GGCA | AAAG | TGTC | GTTT | CTCT |
| TGTG | TGCT | GGTT | CTCA | TAAC |

The proportion of the above motifs was calculated through the following formula:

$${Fraction}_{n , i} = {M}_{i} / {{\sum }_{i = 1}}^{256} {M}_{i}$$

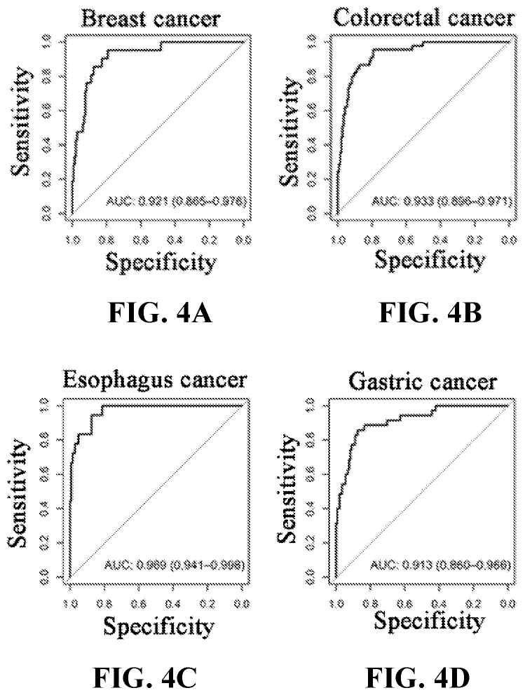

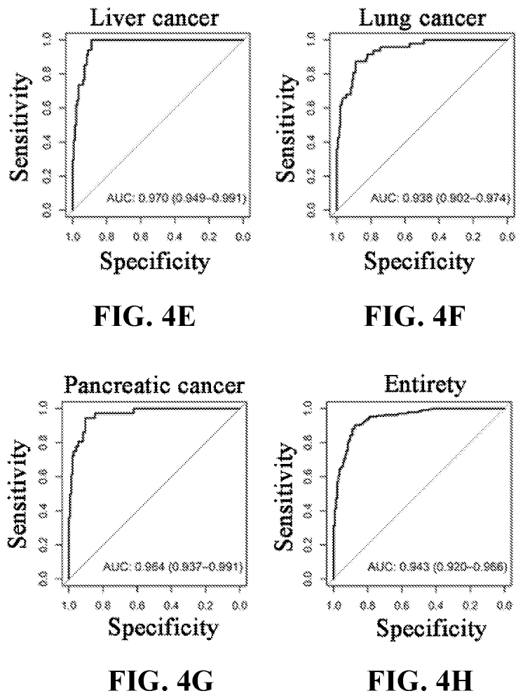

Fraction_{n,i} is the proportion of the i^{th} 4-mer motif of the sample n, and Mᵢ is the number of the i^{th} 4-mer motifs.
4.2 A proportion of 125 characteristic motifs of the healthy person baseline and all cancer samples in the training set were utilized, a caret packet of R language was used for training the SVM model, and the grid search mode was used for selecting the hyper-parameters, and then 10-time cross validation was carried out.
4.3 Classification prediction of healthy persons or cancer patients was performed on each to-be-detected sample in the test set to obtain a predicted value S. As shown in FIG. 4A-4H, the ROC curve area (AUC) of a Motif-SVM classifier for detecting single cancer in the test set reached 0.920-0.966, the AUC performance for detecting all seven cancers reached 0.943, and thus good cancer detection performance was shown.

### 5. Chromosome Instability (CIN) Analysis (CIN-PAscore Analysis Module)

5.1 The number of reads of each semi-arm chromosome after GC correction by the LOESS algorithm was calculated for each sample.
5.2 352 healthy persons in the training set were treated as the baseline samples, and z-score conversion was carried out on the mean value of the reads number of the corresponding semi-arm chromosome of the baseline samples corresponding to the reads number of each semi-arm chromosome of the to-be-detected sample and the standard deviation.
5.3 Five semi-arm chromosomes with the maximum z-score absolute value and the z-score of a semi-arm chromosome corresponding to the baseline sample were selected from the to-be-detected samples, and PAscores were calculated according to a manner (Leary et al., 2012 Sci Transl Med) in the literature. The specific calculation was as follows.

Z_{n,i}=(ARM_{n,i}−MEAN_baselineᵢ)/SD_baselineᵢ
Z_{n,i} is the z-score of the semi-arm chromosome i of the sample n relative to the baseline sample, ARM_{n,i} is the reads number of the semi-arm chromosome i of the sample n, MEAN_baselineᵢ is the mean value of the reads number of the semi-arm chromosome i of the baseline sample, and SD_baselineᵢ is the standard deviation of the reads number of the semi-arm chromosome i of the baseline sample.

The z-scores of five semi-arm chromosomes with the maximum z-score absolute value of the to-be-detected sample n and the z-score of the semi-arm chromosome corresponding to the baseline sample are taken for subsequent analysis:

$$log {P}_{n} = {\sum }_{i = 1}^{5} [ - log ( dt ( {Z}_{n , i} , 3 ) ) ]$$

log Pₙ is a negative value of a logarithm sum of P values of the z-scores of the five semi-arm chromosomes of the sample n in t distribution with the degree of freedom being 3.

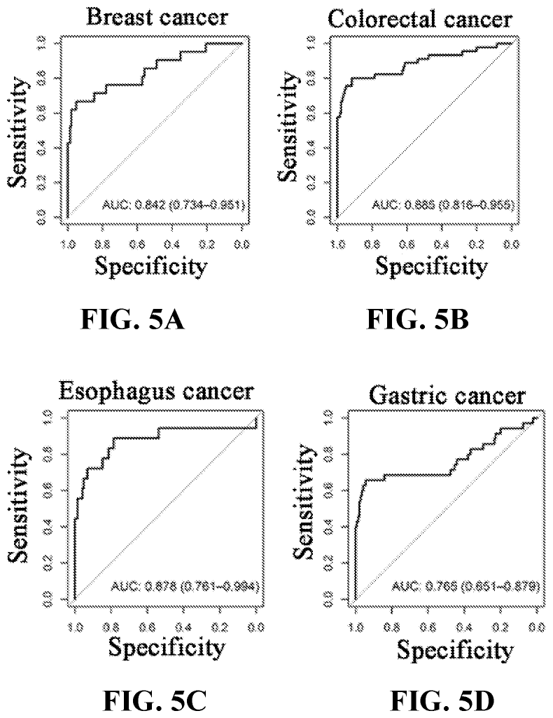

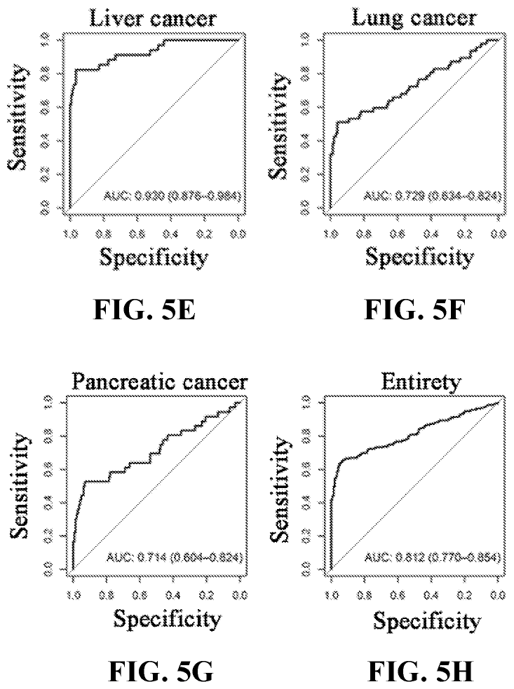

PAscoreₙ=|log Pₙ−MEAN_baseline_{lo□□}|/SD_baseline_{log P}
PAscoreₙ is the PAscore of the sample n, MEAN_baseline_{log P} is the log P mean value of the baseline sample, and SD_baseline_{log P} is the standard deviation of the log P of the baseline sample.
5.4 As shown in FIG. 5A-5H, AUC for detecting single cancer in the test set through a CIN-PAscore algorithm reached 0.770-0.854, and the AUC performance for detecting all seven cancers reached 0.812.

### 6. Construction of Ensemble Model Classifier (SVM-Ensemble Classification Module)

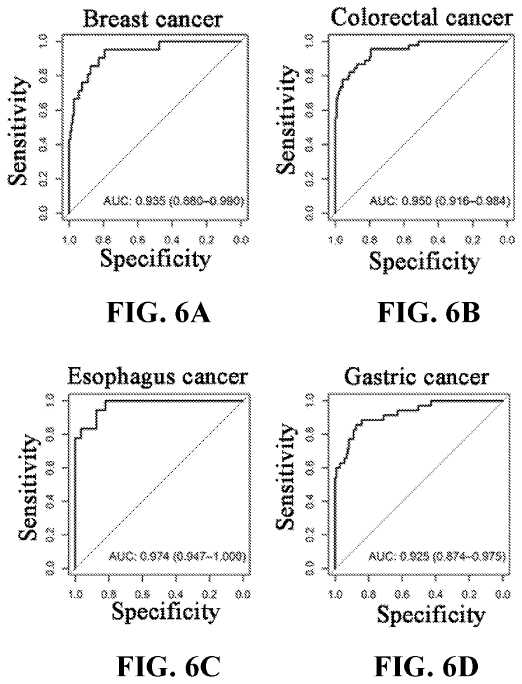

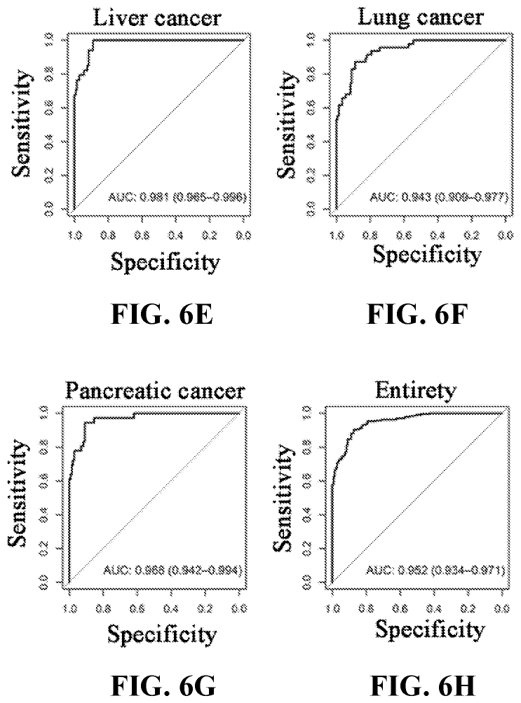

6.1 MD-KNN, FSI-SVM, motif-SVM and CIN-PAscore numerical values (namely the predicted values K, F and S and PAscore) of each sample were treated as characteristics in a training model.
6.2 The LinearSVM model was trained by the caret packet of R language, the hyper-parameters were selected in the grid search mode, and then 10-time cross validation was carried out. Each sample in the test set was predicated through the trained model to obtain a predicted value Z of the sample predicted as single canceration possibility of cancer.
6.3 As shown in FIG. 6A-6H, in the present invention, the AUC of the ensemble model classifier for detecting single cancer in the test set reached 0.934-0.971, the AUC for detecting all seven cancers reached 0.952, and the performance exceeded that of any single genetic or epigenetic characteristic classifier, and thus the superiority of multi-dimensional ensemble analysis of canceration information data relative to single omics was shown.
6.4 As shown in Table 2, in the present invention, the detection sensitivity of the ensemble model classifier for detecting the seven cancers in the test set under 95% specificity was over 60%, the detection sensitivity for early cancer (stage I or stage II) may reach 75%, thus good detection performance for various cancers was shown, and the ensemble model classifier had great potential to be applied for early cancer screening.

TABLE 2
Detection sensitivity of ensemble classification module for verifying
various cancers and various stages in a set under 95% specificity.
Cancer detection performance

| | | Number of | 95% Specificity | |
|---|---|---|---|---|
| | | individuals | Number of individuals | |
| | | analyzed | tested as positive | Sensitivity |
| Type | Healthy | 145 | 8 | — |
| | Cancer | 236 | 173 | 73% |
| | Breast | 21 | 14 | 67% |
| | Colorectal | 45 | 35 | 78% |
| | Esophagus | 18 | 15 | 83% |
| | Gastric | 35 | 22 | 63% |
| | Liver | 34 | 28 | 82% |
| | Lung | 47 | 31 | 66% |
| | Pancreatic | 36 | 28 | 78% |
| Stage | I | 41 | 28 | 68% |
| | II | 34 | 28 | 82% |
| | III | 68 | 43 | 63% |
| | IV | 63 | 45 | 71% |
| | X | 29 | 28 | 97% |

What is claimed is:

1. A detection system of genomic carcinogenesis information based on cell-free DNA, comprising:
a library construction apparatus, configured to convert 5-methylcytosine in cell-free DNA in a to-be-detected sample into 5-formylcytosine and 5-carboxycytosine and convert non-methylated cytosine into uracil by using enzymes to construct a library;
a sequencing apparatus, configured to sequence the constructed library; and
an information analysis apparatus, comprising one or more of the following modules:
a methylation analysis module, configured to analyze methylation information of the cell-free DNA,
a fragment size index analysis module, configured to analyze fragmentation information of the cell-free DNA,
an end motif analysis module, configured to analyze fragmentation information of the cell-free DNA, and
a chromosome instability analysis module, configured to analyze copy number variation information of chromosomes.

2. The system according to claim 1, wherein the information analysis apparatus further comprises an ensemble classification module, configured to perform ensemble on information obtained by the methylation analysis module, the fragment size index analysis module, the end motif analysis module and/or the chromosome instability analysis module.

3. The system according to claim 2, wherein
the methylation analysis module is an MD-KNN analysis module and is configured to divide human reference genome into bins in a non-overlapping sliding window method, calculate a proportion of methylation sites in all CpG sites of each bin, namely a methylation density MD value, and calculate a predicted value K of canceration possibility through a KNN model;
the fragment size index analysis module is an FSI-SVM analysis module and is configured to divide human reference genome into bins in a non-overlapping sliding window method, calculate a proportion of the number of short fragments and the number of long fragments in each bin to obtain a fragment size index FSI value of each sample, and calculate a predicted value F of canceration possibility through an SVM model;
the end motif analysis module is a Motif-SVM analysis module and is configured to calculate a proportion of 5′ end 4-mer motif sequence of a fragment of a sample and calculate a predicted value S of canceration possibility through the SVM model;
the chromosome instability analysis module is a CIN-PAscore analysis module and is configured to calculate a copy number of all semi-arm chromosomes of a sample, and calculate PAscore by performing ensemble on z-scores of five semi-arm chromosomes with the maximum copy number variation of chromosomes corresponding to a healthy human baseline sample; and
the ensemble classification module is an SVM-ensemble classification module and is configured to perform ensemble on the predicted values K, F and S and the PAscore by using a linear SVM model to obtain a final predicted value Z of single canceration possibility.

4. The system according to claim 1, wherein the library construction apparatus comprises:
a plasma cell-free DNA extraction module, configured to extract cell-free DNA from a plasma sample;
an enzyme reaction module, configured to convert 5-methylcytosine in the cell-free DNA into 5-formylcytosine and 5-carboxycytosine, and convert non-methylated cytosine into uracil by using enzymes; and
a PCR reaction module, configured to amplify the cell-free DNA subjected to enzyme reaction by using PCR.

5. The system according to claim 1, wherein the enzymes are TET2 enzyme and APOBEC enzyme.

6. The system according to claim 1, wherein the sequencing apparatus is selected from Illumina Novaseq 6000, Illumina Nextseq500, MGI DNBSEQ-T7 or MGI SEQ-2000.

7. The system according to claim 3, wherein the MD value in the MD-KNN analysis module is calculated through the following formula:
MD_{n,i}=Total_mC_{n,i}/Total_C_{n,i}
wherein MD_{n,i} is the MD value of the i^{th} bin of a sample n, Total_mCᵢ is the total number of all methylated C in the i^{th} bin, and Total_C_{n,i} is the total number of all C in the i^{th} bin.

8. The system according to claim 3, wherein the FSI value in the FSI-SVM analysis module is calculated through the following formula:
FSI_{n,i}=Total_S_{n,i}/Total_L_{n,i}
wherein FSI_{n,i} is the FSI value of the i^{th} bin of a sample n, Total_S_{n,i} is the number of short fragments in the i^{th} bin, and Total_L_{n,i} is the number of long fragments in the i^{th} bin.

9. The system according to claim 3, wherein the proportion of motifs in the motif-SVM analysis module is calculated through the following formula:
$${Fraction}_{n , i} = {M}_{i} / {{\sum }_{i = 1}}^{256} {M}_{i}$$
wherein Fraction_{n,i} is the proportion of the i^{th} 4-mer motif of a sample n, and Mᵢ is the number of the i^{th} 4-mer motifs.

10. The system according to claim 3, wherein the PAscore in the CIN-PAscore analysis module is calculated through the following formula:
Z_{n,i}=(ARM_{n,i}−MEAN_baselineᵢ)/SD_baselineᵢ
wherein Z_{n,i} is the z-score of a semi-arm chromosome i of a sample n relative to the baseline sample, ARM_{n,i} is the reads number of the semi-arm chromosome i of the sample n, MEAN_baselineᵢ is the mean value of the reads number of the semi-arm chromosome i of the baseline sample, and SD_baselineᵢ is the standard deviation of the reads number of the semi-arm chromosome i of the baseline sample;
the z-scores of five semi-arm chromosomes with the maximum z-score absolute value of the to-be-detected sample n and the z-score of the semi-arm chromosome corresponding to the baseline sample are taken for following analysis:
$$log {P}_{n} = {\sum }_{i = 1}^{5} [ - log ( dt ( {Z}_{n , i} , 3 ) ) ]$$
wherein log Pₙ is a negative value of a logarithm sum of P values of the z-scores of the five semi-arm chromosomes of the sample n in t distribution with the degree of freedom being 3; and
PAscoreₙ=|log Pₙ−MEAN_baseline_{log P}|/SD_baseline_{log P}
wherein PAscoreₙ is the PAscore of the sample n, MEAN_baseline_{log P} is the log P mean value of the baseline sample, and SD_baseline_{log P} is the standard deviation of the log P of the baseline sample.

11. The system according to claim 1, wherein the information analysis apparatus comprises a data preprocessing module, configured to convert offline FASTQ data obtained by the sequencing apparatus into a Bam file which can be used by all modules and establish an index.

12. A detection method of genomic carcinogenesis information based on cell-free DNA, performed through the system according to claim 1, comprising:
library construction: converting 5-methylcytosine in cell-free DNA in a to-be-detected sample into 5-formylcytosine and 5-carboxycytosine and converting non-methylated cytosine into uracil by using enzymes to construct a library;
whole-genome sequencing: sequencing the constructed library; and
sequencing information analysis, comprising one or more of the following analysis steps:
methylation analysis: analyzing methylation information of the cell-free DNA,
fragment size index analysis: analyzing fragmentation information of the cell-free DNA,
end motif analysis: analyzing fragmentation information of the cell free DNA, and
chromosome instability analysis: analyzing copy number variation information of chromosomes.

13. The method according to claim 12, wherein the sequencing information analysis further comprises an ensemble classification step of performing ensemble on the information obtained through the methylation analysis, the fragment size index analysis, the end motif analysis and/or the chromosome instability analysis.

14. The method according to claim 13, wherein
the methylation analysis comprises dividing human reference genome into bins in a non-overlapping sliding window method, calculating a proportion of methylation sites in all CpG sites of each bin, namely a methylation density MD value, and calculating a predicted value K of canceration possibility through a KNN model;
the fragment size index analysis comprises dividing the human reference genome into bins in the non-overlapping sliding window method, calculating a proportion of the number of short fragments and the number of long fragments in each bin to obtain a fragment size index FSI value of each sample, and calculating a predicted value F of canceration possibility through an SVM model;
the end motif analysis comprises calculating a proportion of a 5′ end 4-mer motif sequence of a fragment of a sample, and calculating a predicted value S of canceration possibility through the SVM model;
the chromosome instability analysis comprises calculating a copy number of all semi-arm chromosomes of a sample, and calculating PAscore by performing ensemble on z-scores of five semi-arm chromosomes with the maximum copy number variation of chromosomes corresponding to a healthy human baseline sample; and
the ensemble classification comprises performing ensemble on the predicted values K, F and S and the PAscore by using a linear SVM model to obtain a final predicted value Z of single canceration possibility.

15. The method according to claim 12, wherein the library construction comprises:
extracting cell-free DNA from a plasma sample;
enzyme reaction, converting 5-methylcytosine in the cell-free DNA into 5-formylcytosine and 5-carboxycytosine and converting non-methylated cytosine into uracil by using enzymes; and
PCR amplification, amplifying the cell-free DNA subjected to the enzyme reaction by utilizing PCR.

16. The method according to claim 12, wherein the enzymes are TET2 enzyme and APOBEC enzyme.

17. The method according to claim 12, wherein the sequencing is performed by using Illumina Novaseq 6000, Illumina Nextseq500, MGIDNBSEQ-T7 or MGI SEQ-2000.

18. The method according to claim 14, wherein the MD value is calculated through the following formula:
MD_{n,i}=Total_mC_{n,i}/Total_C_{n,i}
wherein MD_{n,i} is the MD value of the i^{th} bin of a sample n, Total_mCᵢ is the total number of all methylated C in the i^{th} bin, and Total_C_{n,i} is the total number of all C in the i^{th} bin;
the FSI value is calculated through the following formula:
FSI_{n,i}=Total_S_{n,i}/Total_L_{n,i}
wherein FSI_{n,i} is the FSI value of the i^{th} bin of the sample n, Total_S_{n,i} is the number of short fragments in the i^{th} bin, and Total_L_{n,i} is the number of long fragments in the i^{th} bin;
the motif proportion is calculated through the following formula:
$${Fraction}_{n , i} = {M}_{i} / {{\sum }_{i = 1}}^{256} {M}_{i}$$
wherein Fraction_{n,i} is the proportion of the i^{th} 4-mer motif of the sample n, and Mᵢ is the number of the i^{th} 4-mer motif;
the PAscore is calculated through the following formula:
Z_{n,i}=(ARM_{n,i}−MEAN_baselineᵢ)/SD_baselineᵢ
wherein Z_{n,i} is the z-score of a semi-arm chromosome i of the sample n relative to the baseline sample, ARM_{n,i} is the reads number of the semi-arm chromosome i of the sample n, MEAN_baselineᵢ is the mean value of the reads number of the semi-arm chromosome i of the baseline sample, and SD_baselineᵢ is the standard deviation of the reads number of the semi-arm chromosome i of the baseline sample;
the z-scores of five semi-arm chromosomes with the maximum z-score absolute value of the to-be-detected sample n and the z-score of the semi-arm chromosome corresponding to the baseline sample are taken for following analysis:
$$log {P}_{n} = {\sum }_{i = 1}^{5} [ - log ( dt ( {Z}_{n , i} , 3 ) ) ]$$
wherein log Pₙ is a negative value of a logarithm sum of P values of the z-scores of the five semi-arm chromosomes of the sample n in t distribution with the degree of freedom being 3; and
PAscoreₙ=|log Pₙ−MEAN_baseline_{log P}|/SD_baseline_{log P}
wherein PAscoreₙ is the PAscore of the sample n, MEAN_baseline_{log P} is the log P mean value of the baseline sample, and SD_baseline_{log P} is the standard deviation of the log P of the baseline sample.

19. The method according to claim 12, wherein the information analysis further comprises data preprocessing, comprising: converting offline FASTQ data obtained by a sequencing apparatus into a Bam file which can be used by all modules and establishing an index.
